(19) United States
(12) Patent Application Publication
(10) Pub. No.: US 2024/0415433 A1
(43) Pub. Date: Dec. 19, 2024

(54) SYSTEMS AND METHODS FOR DETECTING ANXIETY USING EEG

(71) Applicant: NEUMORA THERAPEUTICS, INC., Brisbane, CA (US)

(72) Inventor: Tanya WALLACE, Brisbane, CA (US)

(21) Appl. No.: 18/724,083

(22) PCT Filed: Dec. 30, 2022
(86) PCT No.: PCT/US2022/082610

§ 371 Date: Jun. 25, 2024

Related U.S. Application Data
(60) Provisional application No. 63/295,355, filed on Dec. 30, 2021.

(51) Int. Cl. A61B 5/16; A61B 5/00; A61B 5/374; A61K 45/06

(52) U.S. Cl. CPC A61B 5/165; A61B 5/374; A61B 5/4848; A61B 5/6803; A61B 5/7475; A61K 45/06

(57) ABSTRACT

Methods and systems are provided for evaluating mental health conditions based on a EEG data. According to one implementation, a system for evaluating mental health in a subject includes: a set of EEG sensors configured to output brain wave data collected from the subject. The system also includes a processor, and logic that is integrated with and/or executable by the processor. Moreover, the logic is configured to: receive, by the processor, brain wave data from the set of EEG sensors, the brain wave data corresponding to a treatment administered to the subject, and evaluate, by the processor, the brain wave data to determine an EEG profile over a period of time. The logic is also configured to: determine, by the processor, a mental health condition of the subject using the EEG profile, and output, by the processor via a user interface, an indication of the mental health condition.

### CROSS-REFERENCE TO RELATED APPLICATION

This application claims priority from and benefit of U.S. Provisional Patent Application Ser. No. 63/295,355, filed on Dec. 30, 2021, titled “SYSTEMS AND METHODS FOR DETECTING ANXIETY USING EEG,” which is hereby incorporated by reference herein in its entirety.

### FIELD

The present disclosure relates to diagnosis of mental health disorders using electroencephalography.

### BACKGROUND

The following description includes information that may be useful in understanding the present invention. It is not an admission that any of the information provided herein is prior art or relevant to the presently claimed invention, or that any publication specifically or implicitly referenced is prior art.

Mental health disorders, such as mood disorders are common and debilitating, and can present across multiple related symptom axes, including depressive, anxious, and anhedonia symptoms. Diagnosis of these mental health disorders are typically performed by a healthcare provider using clinical scales implemented based on standard classification systems, such as “The Diagnostic and Statistical Manual of Mental Disorders, 5^{th} edition” (DSM-5), and clinical judgement. Each mental health disorder is diagnosed using a corresponding scale based on a set of symptoms including behavioral, cognitive, affective or physical disturbances, as reported by the patient. Thus, mental health diagnosis is reliant upon subjective assessment by the healthcare provider and self-reported symptoms. As such, the diagnosis is subject to patient and expert bias (e.g., experience, expertise, and/or physical and mental fatigue of the health care provider, among other factors), which impacts diagnostic accuracy of the mental health condition.

### SUMMARY

The disclosed technology is directed to systems and methods for detecting anxiety using Electroencephalography (EEG) sensors. In some implementations, the systems and methods described herein may be used to determine effectiveness of one or more anxiolytic drugs. In some implementations, the systems and methods described herein may be used to determine whether a patient may respond to an anxiolytic medication. In some implementations, the systems and methods described herein may be used to determine a treatment regimen. For example, the systems and methods may be used to determine when another dose of the anxiolytic drug may be beneficial. In some implementations, the systems and methods described herein may be used to determine when a drug's impact is most prevalent. The disclosed technology provides improvements in mental health diagnostics, and further provide improvements in treatment for mental health orders using EEG sensor data.

In order to inform clinical diagnosis and address the issue of subjective assessment mentioned above, some approaches employ Electroencephalography (EEG). EEG involves measuring and recording electrical activity resulting from a plurality of simultaneous neural processes associated with different portions of the brain. An example approach is provided by Shadli, S. M. et al. Right frontal anxiolytic-sensitive EEG ‘theta’ rhythm in the stop-signal task is a theory-based anxiety disorder biomarker. Sci Rep 11, 19746 (2021). https: doi.org 10.1038 s41598-021-99374-x. Therein, Shadli S. M. et al identified a right-frontal goal-conflict-specific EEG rhythmicity (GCSR) occurring in the rodent hippocampal ‘theta’ frequency range (4-12 Hz). The GCSR positively correlated with neuroticism and trait anxiety, and was reduced by all key (non-panicolytic) classes of anxiolytic drugs.

However, the theta pathology discussed in Shadli S. M. et al., may require goal conflict for detection of the GCSR, and thus, may be situation dependent, varying across situations, and across individuals within a situation; As such, sensitivity and accuracy of the GCSR based anxiety diagnosis may be reduced.

In order to at least partially address the above mentioned issues, the inventors herein have identified novel EEG signatures for diagnosing anxiety. In one example, a method of diagnosing anxiety in a subject comprises receiving, via a plurality of EEG sensors, a set of brain wave data. The brain wave data is collected from the subject, and corresponds to a treatment administered to the subject. The brain wave data is evaluated to determine an EEG profile over a period of time. The EEG profile may be based on (i) alpha brain wave activity of the subject and/or (ii) theta brain wave activity of the subject, during the period of time. Moreover, any mental health conditions of the subject may be determined using the EEG profile. Any such mental health conditions may be output using an indication that may be displayed on a user interface. Moreover, anxiety may be diagnosed based on one or more of a frequency, duration, and timing of one or more of alpha brain wave activity and/or theta brain wave activity, e.g., using the EEG profile. In another example, a method includes receiving EEG data from a plurality of EEG sensors. As noted above, the EEG data may correspond to a subject. The EEG data may be processed to determine a mental health condition of the subject (e.g., indicating that the subject has anxiety). This mental health condition of the subject may be based on alpha brain wave activity and/or theta brain wave activity received.

As one example, the inventors have identified frequency range, duration, and timing of a set of brain wave that are effective in diagnosing general anxiety disorder across various individuals. In one example, general anxiety disorder may be diagnosed using systems and methods described herein without depending on goal conflict approaches.

In some implementations, the disclosed technology could be utilized in a patient monitoring system (e.g. digital wrap around) that is useful to determine whether a particular drug is increasing the alpha and theta frequencies of a patient and for what duration. Also, there is potential to administer clinical scales or other tests at various time points around the expected maximal concentration of the drug for an individual patient (based on the EEG profile) to determine when the drug's impact is most prevalent. Finally, the system could be used to indicate to a caregiver or the patient when another dose of the drug may be beneficial.

Additionally, the disclosed technology could be utilized to determine specific patients that need the drug or might respond better to the drug. This may be patients with particularly low alpha or theta band activity.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1A is a block diagram of a mental health evaluation system for evaluating anxiety based on EEG signals, according to an approach of the disclosure;

FIG. 1B is a block diagram of a mental health evaluation system for evaluating anxiety based on EEG signals, according to another approach of the disclosure;

FIG. 2 is a flow chart illustrating an example method for diagnosing anxiety disorder based on EEG signals, according to an approach of the disclosure;

FIG. 3 is a flow chart illustrating an example method for determining a treatment regimen for anxiety disorder, according to an approach of the disclosure;

FIG. 4 is a flow chart illustrating an example method for monitoring a subject for changes in anxiety levels, according to an approach of the disclosure;

FIG. 5 includes a set of graphs, each depicting changes in activity of a brain wave in marmosets at a plurality of time points determined using EEG under no threat conditions and in response to a treatment with a vasopressin 1a (V1a) receptor antagonist.

FIG. 6 includes a set of graphs, each depicting changes in activity of a brain wave in marmosets at a plurality of time points determined using EEG under threat conditions and in response to a treatment with a vasopressin 1a (Va) receptor antagonist.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some approaches, properties such as dimensions, shapes, relative positions, and so forth, used to describe and claim certain approaches of the invention are to be understood as being modified by the term “about.”

### Definitions

As used herein, the term “patient” refers to a person or an individual undergoing evaluation for a health condition and/or undergoing medical treatment and/or care.

As used herein, the term “data modality” or “modality data” refers to representative form or format of data that can be processed and that may be output form a particular type of sensor or processed, manipulated, or captured by a sensor in a particular way, and may capture a particular digital representation of a particular aspect of a patient or other target. For example, video data represents one data modality, while audio data represents another data modality. In some examples, three dimensional video represents one data modality, and two dimensional video represents another data modality.

As used herein, the term “mental health” refers to an individual's psychological, emotional, cognitive, or behavioral state or a combination thereof.

As used herein, the term “mental health condition” refers to a disorder affecting the mental health of an individual, and the term “mental health conditions” collectively refers to a wide range of disorders affecting the mental health of an individual. These include, but not limited to clinical depression, anxiety disorder, bipolar disorder, dementia, attention-deficit/hyperactivity disorder, schizophrenia, obsessive compulsive disorder, autism, post-traumatic stress disorder, anhedonia, and anxious distress.

As used herein, a “subject” means a human or animal. Usually the animal is a vertebrate such as a primate, rodent, domestic animal or game animal. Primates include marmosets, chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some implementations, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. A subject can be male or female.

A subject can be one who has been previously diagnosed with or identified as suffering from or having a condition in need of treatment of a disease or disorder as described herein (e.g., a mental health disorder) or one or more complications related to such a condition, and optionally, have already undergone treatment for a disease or disorder as described herein (e.g., a mental health disorder) or the one or more complications related to a disease or disorder as described herein (e.g., a mental health disorder). Alternatively, a subject can also be one who has not been previously diagnosed as having a disease or disorder as described herein (e.g., a mental health disorder) or one or more complications related to a disease or disorder as described herein (e.g., a mental health disorder). For example, a subject can be one who exhibits one or more risk factors for a disease or disorder as described herein (e.g., a mental health disorder) or one or more complications related to a disease or disorder as described herein (e.g., a mental health disorder) or a subject who does not exhibit risk factors. It follows that a subject may also be referred to as a “patient” in some instances.

A “subject in need” of treatment for a particular condition can be a subject having that condition, diagnosed as having that condition, or at risk of developing that condition.

As used herein, the terms “treat,” “treatment,” or “treating” refer to therapeutic treatments, wherein the object includes preventing, inhibiting, alleviating, reversing, ameliorating, reducing, slowing down, or stopping the progression or severity of a condition(s) and symptom(s) associated a with disorder(s) or disease(s), e.g. a mental health disease such as anxiety disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The term “therapeutically effective amount” as used herein refers to that amount of active compound or pharmaceutical agent (e.g., an anti-viral drug) that elicits the biological or medicinal response in a subject that is being sought by a researcher, veterinarian, medical doctor, or other clinician, which includes preventing, ameliorating or alleviating the symptoms of the disease or disorder being treated. Methods are known in the art for determining therapeutically effective doses for the instant pharmaceutical composition.

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

The present description relates to systems and methods for mental health evaluation using EEG signals. In particular, the inventors have identified increases in alpha wave activity and theta wave activity corresponding with reduction in anxiety produced by anxiolytic compounds, wherein the increase in alpha wave activity occurs at a frequency range of 8.0-12 Hz, and the increase in theta wave activity occurs at a frequency range of 4.0 Hz-8.0 Hz. Accordingly, the systems and methods described herein could be utilized in a patient monitoring system (e.g., digital wrap around) that is useful to determine whether a particular drug is increasing the alpha and theta frequencies of a patient and for what duration. Also, there is potential to administer clinical scales or other tests at various time points around the expected maximal concentration of the drug for an individual patient (based on the EEG profile) to determine when the drug's impact is most prevalent. Finally, the systems and methods could be used to indicate to a caregiver or the patient when another dose of the drug may be beneficial (e.g., based on reduction on alpha and/or theta activity levels below a threshold). Additionally, the system could be utilized to determine specific patients that need the drug or might respond better to the anxiolytic compound. This may be patients with particularly low frequencies of occurrence of alpha and/or theta waves. According to one implementation, the systems and methods described herein may be utilized as a part (e.g., step) of determining whether one or more Vasopressin receptor antagonists, e.g., such as BTRX-323511 and/or similar drugs, are impacting the alpha and theta frequencies of a patient. If so, these systems and methods are also able to determine the duration, amplitude, etc., that the antagonists (e.g., such as BTRX-323511) are impacting the alpha and theta frequencies of the patient.

According to one implementation, a system for evaluating mental health in a subject includes: a set of EEG sensors configured to output brain wave data collected from the subject. The system also includes a processor, and logic that is integrated with and/or executable by the processor. Moreover, the logic is configured to: receive, by the processor, brain wave data from the set of EEG sensors, the brain wave data corresponding to a treatment administered to the subject, and evaluate, by the processor, the brain wave data to determine an EEG profile over a period of time. The EEG profile is based on (i) alpha brain wave activity of the subject and/or (ii) theta brain wave activity of the subject, during the period of time. The logic is also configured to: determine, by the processor, a mental health condition of the subject using the EEG profile, and output, by the processor via a user interface, an indication of the mental health condition.

Another implementation involves a method of treating, reducing the likelihood of developing, reducing the severity of, or ameliorating the symptoms of a mental health disorder in a subject that has been administered a dosage of an anxiolytic compound. The method includes determining whether the patient is a responder to the anxiolytic compound based on alpha brain wave activity and/or theta brain wave activity. The alpha brain wave activity and/or the theta brain wave activity is determined based on EEG data output from a set of EEG sensors coupled to the subject. Moreover, the method further includes determining a treatment regimen for the subject to either continue, modify or discontinue treatment with the anxiolytic compound based on whether the patient is determined to be a responder to the anxiolytic compound.

Yet another implementation involves a method for mental health evaluation of a subject. The method includes receiving EEG data from one or more electrodes, the EEG data corresponding to the subject, and processing the EEG data to determine an indication of whether the subject responds to vasopressin receptor antagonists.

### System

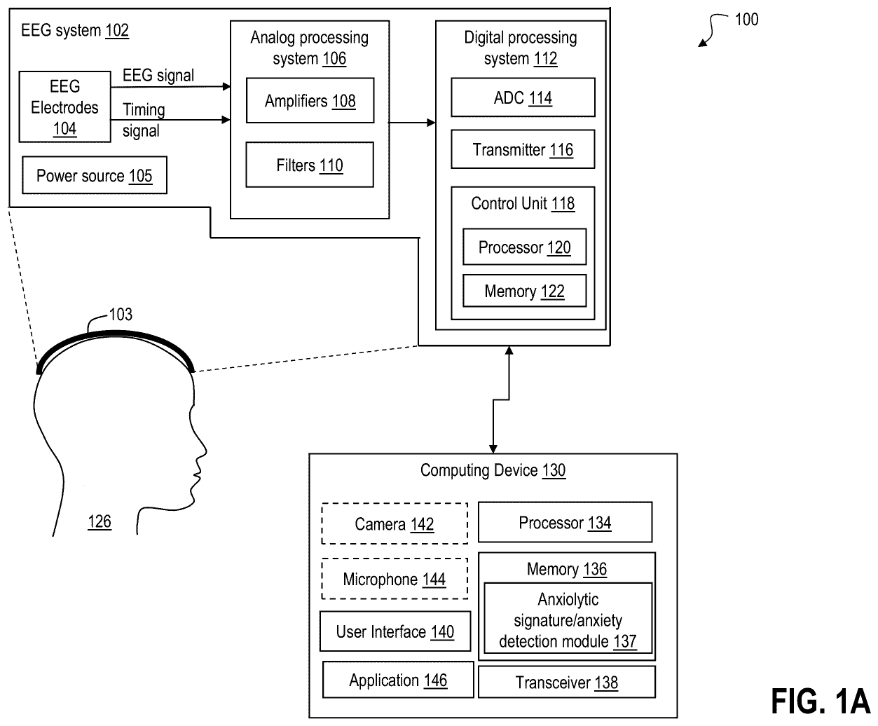

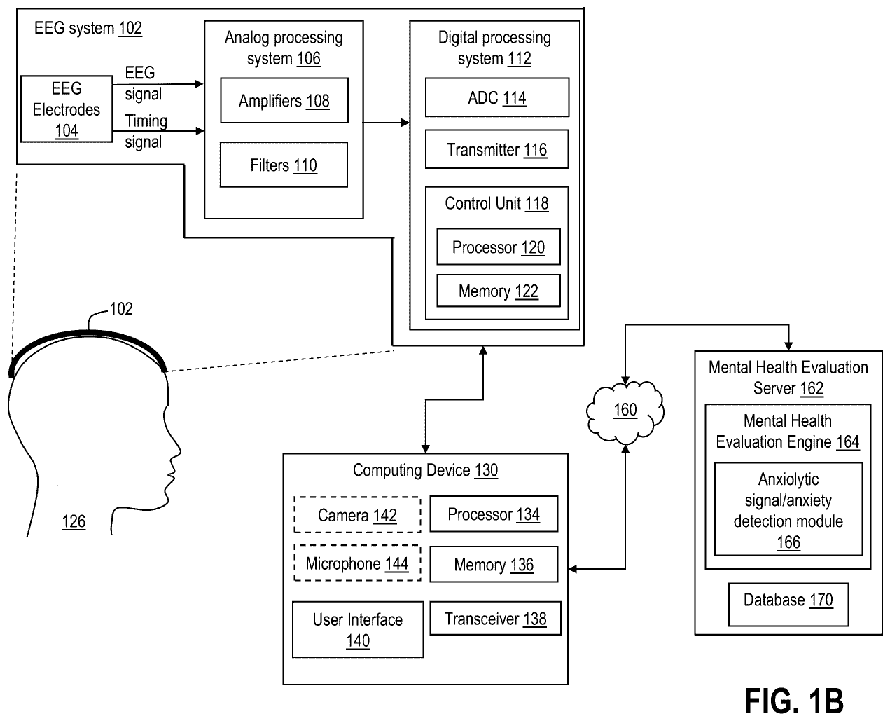

FIG. 1A is a block diagram of an example mental health evaluation system 100. The mental health evaluation system 100 can be used to evaluate the mental health in a subject. The mental health evaluation system 100 comprises an EEG system 102 configured to acquire (e.g., collect) brain wave data from a subject 126. The EEG system 102 comprises a plurality of EEG electrodes 104. The plurality of electrodes 104 In one example, processing system 900 for use with the example headset 100. are coupled, for example, to a headset 103 to be worn on a head of the subject 126. In one example, the headset 103 includes one or more bands to be worn on a head of the subject and the plurality of electrodes 104 that are positioned over the head of the subject. The headset 103 is configured such that the plurality of electrodes is engaged with the scalp of the subject when worn by the subject. The electrodes 104 are conductive electrodes and when positioned on the scalp, measure the small electrical potentials that arise outside of the head due to neuronal action within the brain. In one example, the plurality of electrodes may be configured according to the 10-20 System of electrode placement. In some implementations, the electrodes 104 may include EEG sensors.

In one example, the EEG system 102 is configured as a wearable device. For example, the EEG system 102 may be configured for long term ambulatory use, short-term rapid use (e.g., for a duration after treatment with a medication for mental health disorder), or a combination thereof. In another example, the EEG system 102 may be a clinical grade EEG system, configured for use in clinical environments.

The EEG system 102 further comprises an analog processing system 106 including one or more amplifiers 108 and one or more filters 110 for processing the electrical signals from the plurality of electrodes 102. For example, the amplifier is used to amplify the signal to a more detectable level. In addition, the filters 110 are used to remove noise from the signal. The filters 110 may also be used as a bandpass filter to pass one or more frequency bands and/or manipulate select frequency bands depending on the desired processing and/or analysis. Further, the electrodes 104 are communicatively coupled to the analog processing system 106 via a communications link, which may be wired, wireless, or a combination thereof. With respect to the present description, a “wireless” connection may include any type of computer network that uses wireless data connections between network nodes, e.g., such as WiFi, Bluetooth, cellular network(s), etc. Moreover, a “wired” connection may include any type of physical electrical connection, e.g., cable(s), fiber-optic link(s), wire(s), etc. The amplified and filtered electrical signals from the analog processing system 106 may be transmitted to a digital processing system 112 via a communication link, which may be a wired, a wireless, or a combination thereof. The digital processing system 112 includes an analog-to-digital converter 114, a transmitter 116, and a control unit 118 including a processor 120 and memory 122.

The control unit 118 communicates with the ADC 114 to process information (e.g., data, requests, commands, instructions, files, etc.,) received by the transmitter 116. Similarly, the transmitter 116 may be used to transmit (e.g., send and/or receive) information to other locations in system 100 and/or elsewhere. For instance, the transmitter 116 may be able to send information to and/or receive information from computing device 130 over a network (e.g., see communication network 160 of FIG. 1B). Any one or more of the components in FIG. 1A may be connected to a network depending on the implementation. The network may be of any type, e.g., depending on the desired approach. For instance, in some approaches the network is a WAN, e.g., such as the Internet. However, an illustrative list of other network types which network may implement includes, but is not limited to, a LAN, a PSTN, a SAN, an internal telephone network, etc.

The analog-to-digital converter 114 converts the analog signals received at the electrodes 104 to digital signals. In some examples, the analog-to-digital converter 114 comprises multiple A-D converters located to service individual or sets of the electrodes to convert the signals as close to the source as possible, which may further reduce interference.

The digital processing system 112 also includes hardware and/or software to execute Fast Fourier Transform (FFT) calculations, coherence measurements and/or custom adaptive filtering.

In one example, the transmitter 116 communicates the data at any stage of processing to the control unit 118 and/or from the control unit 118 or ADC 114 to computing device 130. Data transmission may be implemented by Bluetooth transmission, wi-fi transmission, ZIGBee transmission and/or encryption before transmission. In one example, a database may store all data gathered streams. The streams can be buffered for streaming or stored on-board (i.e., at the headset) for periodic or aperiodic uploads during, for example, low-activity periods.

While example manner of implementing the system 100 has been illustrated in FIG. 1A, one or more of the elements, processes and/or devices illustrated in FIG. 1A may be combined, divided, re-arranged, omitted, eliminated and/or implemented in any other way. More generally, the example system 100 of FIG. 1A could be implemented by one or more analog or digital circuit(s), logic circuits, programmable processor(s), application specific integrated circuit(s) (ASIC(s)), programmable logic device(s) (PLD(s)) and/or field programmable logic device(s) (FPLD(s)). Further still, the example system 100 of FIG. 1A may include one or more elements, processes and/or devices in addition to, or instead of, those illustrated in FIG. 1A, and/or may include more than one of any or all of the illustrated elements, processes and devices.

### Example Computing Device(s)

Mental health evaluation system 100 includes a computing device 130 for receiving brain wave data acquired via the EEG system 102. In other words, the brain wave data may be collected from a subject by one or more sensors in the EEG system 102. The computing device 130 may be any suitable computing device, including a computer, laptop, mobile phone, etc. The computing device 130 includes one or more processors 120, one or more memories 122, and a user interface 140 for receiving user input (e.g., touch on a screen, voice, gesture, thought, etc.) and/or presenting information to a user (e.g., on a physical screen, projecting onto a surface, rendering a scene in a virtual environment, etc.).

In one implementation, the computing device 130 may be configured as a mobile device and may include an application, which represent machine executable instructions in the form of software, firmware, or a combination thereof. The components identified in the application may be part of an operating system of the mobile device or may be an application developed to run using the operating system. In one example, application may be a mobile application. The application may also include web applications, which may mirror the mobile application, e.g., providing the same or similar content as the mobile application. In some implementations, the application may be used to initiate brain wave data acquisition for mental health evaluation of a subject. Further, in some examples, the application may be configured to monitor a quality of data acquired from each modality, and provide indications to a user regarding the quality of data.

The application may be used for remote mental health evaluation as well as in-clinic mental health evaluation. In some implementations, the application may include a patient interface and may assist a patient in acquiring modality data for mental health evaluation. As a non-limiting example, the patient interface may include options for activating a camera 142 and/or microphone 144 that are communicatively coupled to the computing device and/or integrated within the computing device. In some examples, the camera 142 and microphone 144 may be used to acquire video and audio data respectively, which may be used for informing mental health evaluation.

In one example, the computing device 130 may receive data from a storage device which stores the data generated by these modalities. In another implementation, the computing device 130 including processor and memory storing instructions for evaluating mental health based on EEG, may be disposed at a device (e.g., edge device, server, etc.) communicatively coupled to a computing system that may receive data from the plurality of EEG sensors and/or systems, and transmit the plurality of EEG data to the device for further processing. It follows that in some implementations, brain wave data may be received from a plurality of EEG sensors that are configured to detect and record brain wave data (among other types of data) from a subject. Accordingly, the EEG sensors may be coupled to, positioned adjacent to, etc., the subject. In one example, a desired number of EEG sensors may be included in the headset 103 such that they are able to detect and record brain waves of a subject while wearing the headset 103.

The computing device 130 further includes a user interface 140. User interface may be a user input device, and may comprise one or more of a touchscreen, a keyboard, a mouse, a trackpad, a motion sensing camera, and other device configured to enable a user to interact with and manipulate data within the processor 134 and/or memory 136.

The processor 134, non-transitory memory 136, and/or user interface 140 may be disposed in a shared enclosure, or may be peripheral display devices and may comprise a monitor, touchscreen, projector, or other display device known in the art, which may enable a user to view modality data, and/or interact with various data stored in non-transitory memory 136.

In one example, memory 136 may include instructions that when executed causes the processor 134 to receive the brain wave data (also referred to as EEG data) via a transceiver 138 and further, pre-process the plurality of EEG data. In some examples, pre-processing the plurality of EEG data may include filtering each of plurality of data modalities to remove noise. In some implementations, the brain wave data (e.g., EEG data) may be transmitted to a mental health evaluation server. For instance, FIG. 1B includes a network 160 that connects computing device 130 to a mental health evaluation server 162. The server 162 and/or network 160 may thereby effectively become part of the system 100. The server 162 may be configured to receive the plurality of data modalities from the computing device 130 via the network 160. However, any desired type of information (e.g., brain wave data, metadata, EEG data, instructions, requests, etc.) may be sent between locations of the system and/or other locations that may be connected to the network 160. The network 160 may be wired, wireless, or various combinations of wired and wireless.

The server 162 may include a mental health evaluation engine 164 for performing mental health condition analysis of a subject. In one example, the mental health evaluation engine 164 includes an anxiolytic signature/anxiety detection module 166 configured for detecting anxiety signatures and/or anxiolytic signatures from the brain wave data collected from a subject (e.g., by one or more EEG sensors). In some examples, the computing device 130 may include the anxiolytic signature/anxiety detection module and the EEG signal analysis and mental health evaluation may be performed at the computing device 130. According to an example, which is in no way intended to be limiting, the anxiolytic signature/anxiety detection module may be used to determine the effectiveness of a V1a receptor antagonist. More specifically, in some implementations the anxiolytic signature/anxiety detection module may be used to determine whether one or more Vasopressin receptor antagonists, e.g., such as BTRX-323511 and/or similar drugs, are impacting the alpha and theta frequencies of a patient. If so, the anxiolytic signature/anxiety detection module may also be able to determine the duration, amplitude, etc., that the antagonists (e.g., such as BTRX-323511) are impacting the alpha and theta frequencies of the patient. This information provides valuable insight as to how the antagonists are affecting the patient, and controlled procedures may be implemented to conduct a variety of tests, e.g., as described in any of the approaches herein.

With continued reference to FIG. 1B, the server 162 may include a database 170 for storing the plurality of EEG data for each patient. In some examples, the database may also store plurality of training and/or validation datasets for training and/or validating an anxiolytic signature/anxiety detection model of the anxiolytic signal/anxiety detection module 166 for performing mental health evaluation. Further, the mental health evaluation output from the anxiolytic signal/anxiety detection module may be stored at the database 170. Additionally, or alternatively, the mental health evaluation output may be transmitted from the server to the computing device, and displayed and/or stored at the computing device 130.

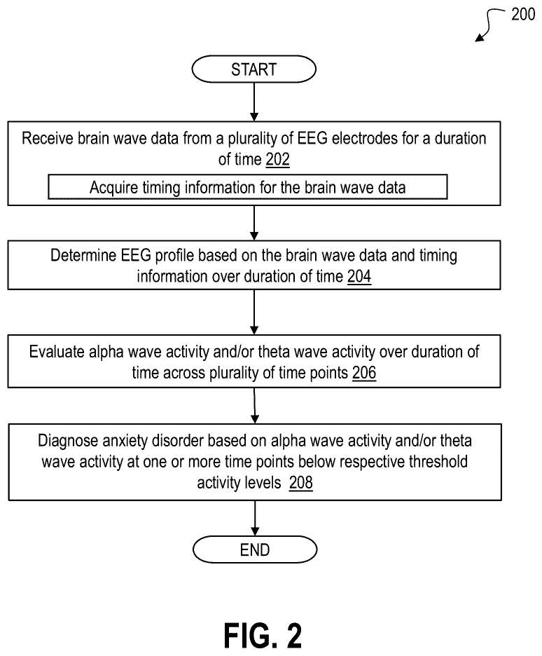

Referring now to FIG. 2, a flow chart of a high-level method 200 for evaluating a mental health condition of a subject is illustrated in accordance with one implementation. This determination may be made based on brain wave data received from a plurality of EEG electrodes (e.g., see EEG electrodes 104 of FIGS. 1A-1B).

The method 200 may be executed by an EEG system processor, such as processor 120; a processor, such as processor 134 of computing device; or one or more processors of mental health evaluation server 164 or a combination thereof. In some examples, the processor executing the method 200 may include a trained machine learning model, trained to classify and/or regress one or more mental health conditions, including but not limited to depression, anxiety disorder, and anhedonic conditions using EEG data.

At 202, the method 200 includes receiving brain wave data from a plurality of EEG sensors. Further, in some examples, timing information may be acquired for the brain wave data. It follows that the timing information is preferably correlated with the brain wave data somehow. For example, the timing information may be a running clock that is synched with the brain wave data received from the plurality of EEG sensors. In some implementations, this synchronization may be achieved by comparing metadata included in the brain wave data and timing information.

Next, at 204, the method 200 includes determining EEG profile based on the brain wave data and timing information over a duration of time. For example, the EEG profile may be based on changes in the alpha wave, beta wave, gamma wave 1, gamma wave 2, delta, etc., as well as changes in theta at various points in time over a given period of time. As a non-limiting example, in marmosets, alpha waves may be determined based on a frequency range of about 8.0-12 Hz, beta waves may be determined based on a frequency range of about 12.0-30.0 Hz, gamma 1 waves may be determined based on frequency range of about 30.0-55.0 Hz, gamma 2 waves may be determined based on a frequency range of about 55.0-100 Hz, delta waves may be determined based on frequency range of about 0.5-4.0 Hz, and theta waves may be determined based on frequency range of about 0.5-4.0 Hz. In one example, the brain wave data may be acquired in the absence of any threat stimulus at a plurality of points in time.

Next, at 206, the method 200 includes evaluating alpha wave activity and/or theta wave activity. The alpha and/or theta wave activity may be evaluated over one or more periods of time that include any desired number of points of interest. It follows that the alpha and/or theta wave activity may be evaluated using any desired processes.

Next, at 208, the method includes diagnosing anxiety disorder. This diagnosis is based on alpha wave activity and/or theta wave activity at one or more time points below respective threshold activity levels. In particular, the inventors have identified that alpha wave activity and theta wave activity increase contemporaneously with reduction in anxiety. Accordingly, in one example, EEG signatures in the alpha frequency range and theta frequency range may be used to diagnose anxiety that may be treatable by anxiolytic medications that increase alpha waves and theta waves. For example, very low activity in the alpha frequency range and in the theta frequency range may provide an indication of anxiety disorder that may be treatable by the anxiolytic medications that increase alpha and theta waves. The overall efficacy, ideal dosage information, etc., may further be determined for these medications (e.g., such as BTRX-323511) by evaluating how the alpha and theta frequencies of the patient were impacted during/after treatment. This information provides valuable insight as to how the medications are affecting the patient, and controlled procedures may be implemented to conduct a variety of tests on medications, e.g., as described in any of the approaches herein.

Responsive to diagnosing anxiety disorder based on alpha wave activity and/or theta wav activity, the diagnosis may be output via a user interface, such as user interface of the computing device 130.

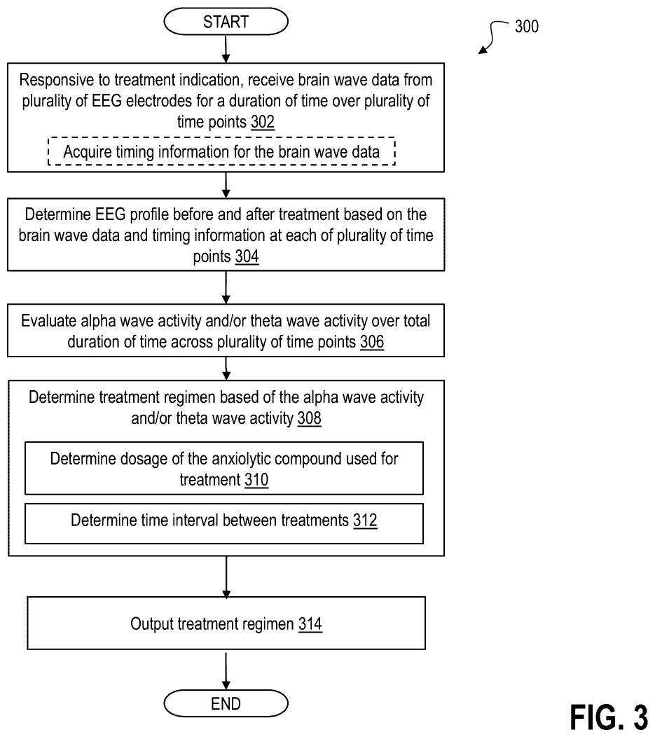

Turning to FIG. 3, a flow chart of a high-level method 300 for determining a treatment regimen for anxiety disorder in a subject is illustrated in accordance with one implementation. This determination may be made using brain wave data received from a plurality of EEG electrodes (e.g., see EEG electrodes 104 of FIGS. 1A-1B).

The method 300 may be performed in accordance with any of the implementations depicted in and/or described in relation to FIGS. 1A-1B, among others. Of course, more or less operations than those specifically described in FIG. 3 may be included in method 300, as would be understood by one of skill in the art upon reading the present descriptions.

Each of the operations of the method 300 may be performed by any suitable component of the operating environment using known techniques and/or techniques that would become readily apparent to one skilled in the art upon reading the present disclosure. For example, method 300 may be executed by an EEG system processor, such as processor 120; a processor, such as processor 134 of computing device; one or more processors of mental health evaluation server 164; etc.; and/or a combination thereof. The processor, e.g., processing circuit(s), chip(s), and/or module(s) implemented in hardware and/or software, and preferably having at least one hardware component may be utilized in any device to perform one or more steps of the method 300. Illustrative processors include, but are not limited to, a central processing unit (CPU), an application specific integrated circuit (ASIC), a field programmable gate array (FPGA), etc., combinations thereof, or any other suitable computing device known in the art.

In some examples, the processor executing the method 300 may include a trained machine learning model, trained to classify and/or regress one or more mental health conditions, including but not limited to depression, anxiety disorder, and anhedonic conditions using EEG data and determine a treatment regimen based on the EEG response profile of the subject in response to a treatment with an anxiolytic compound. As noted herein, one example compound that may be evaluated using any of the approaches included herein and/or used for treatment include Vasopressin (V1a) receptor antagonists, e.g., such as those described in U.S. Pat. No. 10,538,530, which is incorporated by reference in its entirety. Accordingly, in some implementations, method 300 involves treating, reducing the likelihood of developing, reducing the severity of, or ameliorating the symptoms of a mental health disorder in a subject that has been administered a dosage of an anxiolytic compound, e.g., as will be described in further detail below.

Referring still to FIG. 3, operation 302 of method 300 includes receiving brain wave data from a plurality of EEG sensors at a plurality of points in time (e.g., “time points”). In particular, brain wave data may be acquired at the plurality of points in time after treating the subject with an anxiolytic compound. Further, in some examples, timing information may be acquired for the brain wave data. As noted above, the timing information is preferably correlated with the brain wave data. For example, the timing information may be a running clock that is synched with the brain wave data received from the plurality of EEG sensors. In some implementations, this synchronization may be achieved by comparing metadata included in the brain wave data and timing information.

Operation 304 further includes determining EEG profile based on the brain wave data and timing information over a duration of time before and after treatment with the anxiolytic compound.

At operation 306, the method 300 includes evaluating alpha wave activity and theta wave activity at a plurality of time points. As noted above, alpha and/or theta wave activity may be evaluated at any desired point in time before, during, and/or after treatment with the anxiolytic compound. Accordingly, operation 306 may more generally include determining whether the patient is a responder to the anxiolytic compound based on alpha brain wave activity and/or theta brain wave activity. The alpha brain wave activity and/or theta brain wave activity may be determined based on EEG data output from a set of EEG sensors coupled to the subject, e.g., according to any of the implementations included herein.

Next, at operation 308, the method 300 includes determining a treatment regimen with the anxiolytic compound. The treatment regimen may be determined using alpha and theta wave activity recorded in response to (following) a treatment with the anxiolytic compound being tested. For example, instances (e.g., points in time) where the anxiolytic compound caused increases in the alpha wave and/or the theta wave may be determined and used for further evaluation of the anxiolytic compound with respect to the given subject. Additionally, instances where the anxiolytic compound caused the alpha wave and/or the theta wave to decrease (e.g., after an increase) below respective threshold activity levels may be determined. These instances may also be used for further evaluation of the anxiolytic compound with respect to the given subject. For instance, dosage interval (312) may be determined based on detecting a decrease after an increase in alpha and/or theta wave activity. Further, the dosage amount (310) may be based on an amount of anxiolytic compound required to elicit alpha wave activity and/or theta wave activity to increase above respective predetermined threshold activity levels. These predetermined threshold activity levels may be higher than a standard threshold activity level. The predetermined threshold activity levels may be set by a user, a medication manufacturer, industry standards, input from a physician, results from machine learning procedures, etc.

Method 300 also includes outputting the treatment regimen. See operation 314. This treatment regimen may be developed for the subject to indicate whether to continue, modify, or discontinue use of the anxiolytic compound, based on whether the patient is identified as being a responder to the anxiolytic compound. In other words, a treatment regimen is determined for the subject, where the treatment regimen indicates whether the subject should maintain or change use of an anxiolytic compound based on how the subject reacts to the anxiolytic compound being administered to them.

In some approaches, the treatment regimen is output by being displayed on a user interface, e.g., such as user interface 140 of FIGS. 1A-1B above. In other approaches, the treatment regimen may be displayed on a user device after being sent over a network, stored at a memory location that maintains patient data, transformed into a different format (e.g., encrypted, compressed, etc.) and sent to a predetermined location, etc. In still other approaches, the treatment regimen may actually be encrypted and stored in memory as mentioned above, while a notification indicating that the treatment regimen is available for review is sent to a medical professional. The medical professional may thereby log in to a secure network over a network, at a terminal, etc., to access the treatment regimen, thereby minimizing the risk of the patient's data being exposed.

In this way, EEG signals may be used to determine whether a particular drug is increasing the alpha and theta frequencies of a patient and for what duration. Accordingly, a treatment regimen (dosage and interval between doses) may be determined.

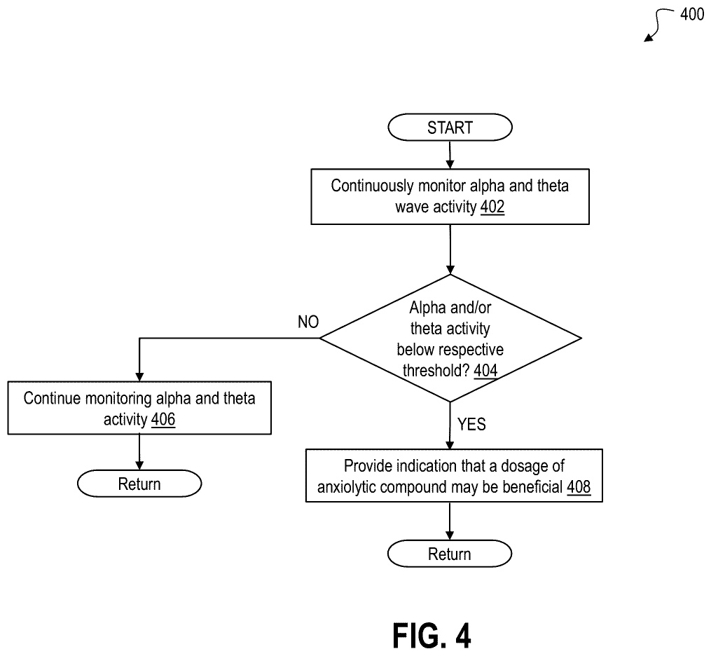

Next, FIG. 4 shows a high-level flow chart illustrating an example method 400 for providing one or more indications to a user based on alpha and theta activity level response after treatment with an anxiolytic compound. As noted above, the method 400 may be performed in accordance with any of the implementations depicted in and/or described in relation to FIGS. 1A-1B, among others.

Each of the steps of the method 400 may be performed by any suitable component of the operating environment using known techniques and/or techniques that would become readily apparent to one skilled in the art upon reading the present disclosure. For example, in various implementations, the method 400 may be partially or entirely performed by a controller, a processor, etc., or some other device having one or more processors therein. The processor, e.g., processing circuit(s), chip(s), and/or module(s) implemented in hardware and/or software, and preferably having at least one hardware component may be utilized in any device to perform one or more steps of the method 400. Illustrative processors include, but are not limited to, a central processing unit (CPU), an application specific integrated circuit (ASIC), a field programmable gate array (FPGA), etc., combinations thereof, or any other suitable computing device known in the art.

At 402, the method 400 includes continuously monitoring one or more EEG profiles. Each of the EEG profiles may include alpha wave activity and/or theta wave activity of a subject (e.g., “patient”). In some approaches, the alpha wave activity and/or theta wave activity may be determined based on brain wave data received from various EEG sensors, e.g., as discussed above with respect to FIG. 2.

Operation 404 of method 400 further includes determining whether the alpha wave activity and/or theta wave activity has decreased below respective threshold activity levels. In other words, operation 404 includes determining whether the alpha wave activity has fallen below a first threshold, in addition to determining whether the theta wave activity has fallen below a second threshold. In some instances, it is desirable to also determine whether a decrease in alpha and/or theta wave activity follows (e.g., comes after) an initial increase in wave activity. For instance, this increase in alpha and/or theta wave activity may be in response to the subject receiving treatment with an anxiolytic compound, e.g., such as BTRX-323511.

Method 400 proceeds to operation 408 in response to determining that alpha wave activity and/or theta wave activity has decreased below respective threshold activity levels. There, operation 408 includes providing an indication (e.g., to a caregiver, a patient etc.) that another dosage of anxiolytic compound may be beneficial. In other words, method 400 proceeds to operation 408 in response to determining that the effects of a last dosage is wearing off, and the subject would benefit from receiving another dosage of the medication (e.g., such as BTRX-323511) being evaluated. However, method 400 proceeds to operation 406 in response to determining that the alpha wave activity and/or theta wave activity has not decreased by predetermined amounts. At 406, the method 400 continues to monitor alpha and/or theta wave activity. As a result, method 400 may be able to evaluate various aspects of testing a medication, e.g., including the ability to identify when subsequent dosages should be administered to the subject.

### Examples

The following examples are provided to better illustrate various aspects of the claimed invention and are not intended to be interpreted as limiting the scope of the invention in any way. To the extent that specific materials or steps are mentioned, it is merely for purposes of illustration and is not intended to limit the invention. One skilled in the art may develop equivalent means or reactants without the exercise of inventive capacity and without departing from the scope of the invention. Moreover, any of these examples may be implemented in any of the implementations described above. For example, EEG signatures may be recorded and/or evaluated using a system as described in any of the implementations above with respect to FIGS. 1A-1B.

Various ones of the examples include a novel EEG signature observed in the alpha and theta frequency range of the EEG observed in non-human primates in relation to a V1a receptor antagonist, with or without a threat stimulus. Three studies were performed with marmosets. Two of these studies were behavioral and demonstrated that two different V1a receptor antagonists were anxiolytic for the animals being tested. A third study included animals instrumented with EEG electrodes and demonstrated that the same dose of the V1a receptor antagonist that produced anxiolytic effects in the first two studies, also increased the alpha and theta frequency bands at the maximal concentration of the compound in the plasma. In addition, following a threat stimulus, the alpha and theta frequency bands further increased with the application of a V1a antagonist.

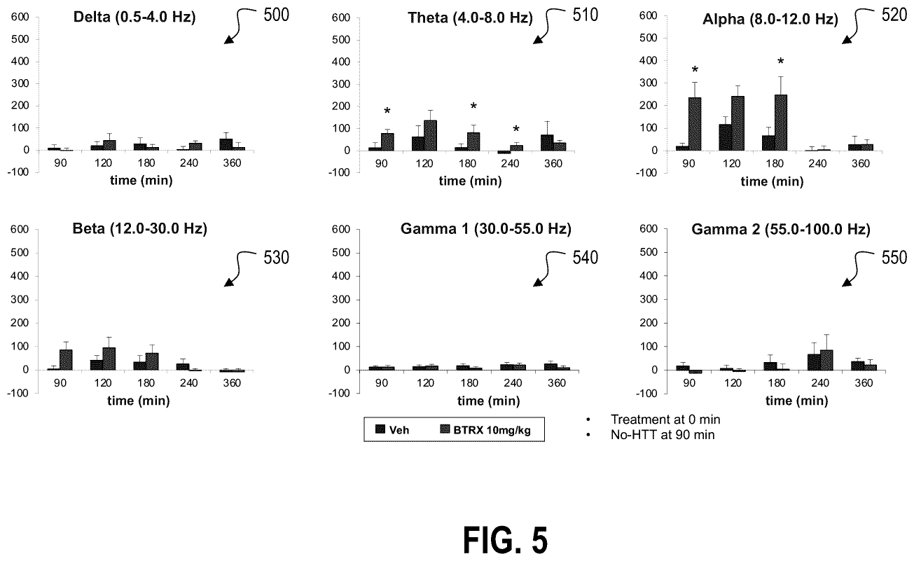

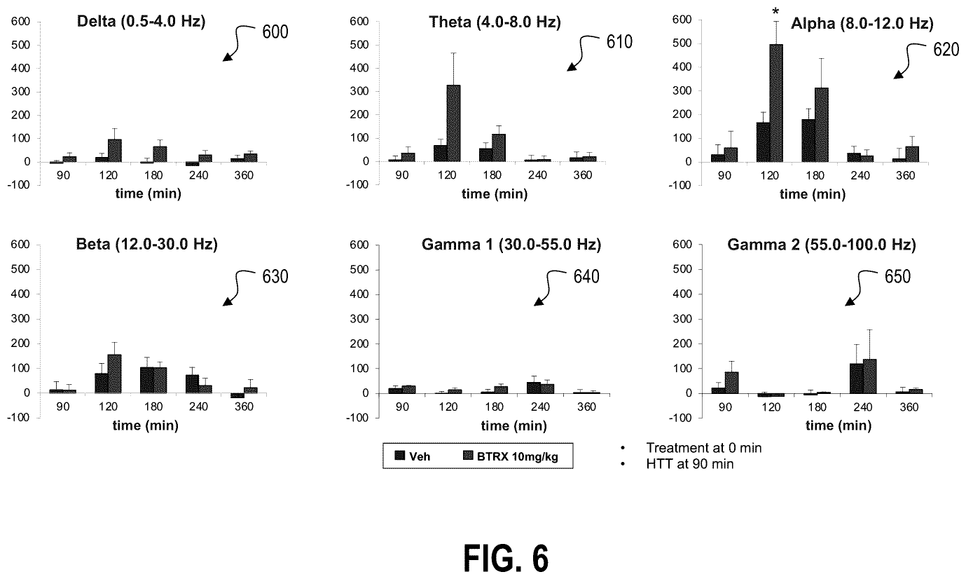

According to some examples, the V1a antagonist may be BTRX-323511. Accordingly, BTRX-323511 may be administered to a subject who is being evaluated, e.g., using EEG electrodes and/or any aspects of system 100 (e.g., as depicted in FIGS. 1A-1B). Looking specifically now to FIG. 5, a set of graphs 500, 510, 520, 530, 540, 550 are shown. Each of the graphs indicate brain wave activity in marmosets over time in the absence of a threat condition, and in response to BTRX-323511 being administered to the marmosets. Similarly, FIG. 6 includes a set of graphs 600, 610, 620, 630, 640, 650, each indicating brain wave activity of marmosets over time, while experiencing a threat stimulus and in response to the V1a receptor antagonist (BTRX-323511) being administered to the marmosets.

The waves in graphs 520 and 510 show that the alpha and theta wave activity increases in response to treatment with V1a receptor antagonist. While the data included in graphs 520 and 510 was captured in the absence of a threat condition, similar results are achieved in the presence of threat conditions as well. For instance, alpha and theta waves depicted in graphs 620 and 610, respectively, show that the alpha and theta wave activity increases in response to treatment with V1a receptor antagonist, even in situations where a threat condition is present. This can be seen in the data plotted between 0 and 180 minutes in both FIGS. 5 and 6 following treatment using BTRX-323511. During this period, the alpha and theta wave activities increase in response to the BTRX-323511 treatment. The increase in alpha and theta wave activities correlates with increased concentration of the compound in plasma. This further indicates that anxiolytic effects correspond to increases in alpha and theta wave frequencies.

With respect to the present description, it should be noted that a BTRX-323511 treatment may be administered in a number of different ways, e.g., depending on the particular implementation. For instance, some implementations the medication is a pharmaceutical composition that includes an active compound having the BTRX-323511, together with at least one pharmaceutically acceptable carrier, diluent, excipient, etc. Depending on the approach, the active compound may be mixed with a carrier, diluted by a carrier, enclosed in a carrier, etc. Moreover, the carrier may be in the form of an ampoule, capsule, sachet, paper, etc., or any other desired type of container.

For implementations in which the active compound is mixed with a carrier, or when the carrier serves as a diluent, the carrier may be in a solid, semi-solid, or liquid state. The carrier may also act as a vehicle, excipient, or medium for the active compound. The active compound is adsorbed on a granular solid carrier in some approaches. Examples of suitable carriers include, but are in no way limited to, water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, polyoxyethylene, hydroxymethyl cellulose, and polyvinylpyrrolidone. Similarly, the carrier or diluent can include any sustained release material known in the art, such as glyceryl monostearate or glyceryl distearate, alone or mixed with a wax.

The route of administering the medication to a patient (e.g., subject) can be any route which effectively transports the active compound to the appropriate and/or desired site of action. These include oral, nasal, pulmonary, buccal, subdermal, intradermal, transdermal, or parenteral, e.g., rectal, depot, subcutaneous, intravenous, intraurethral, intramuscular, intranasal, ophthalmic solution, an ointment, etc. However, the oral route is preferred in some implementations.

It should also be noted that the various studies described herein that pertain to evaluating how marmoset respond to various medications may be translated to humans. For instance, corresponding alpha and theta wave frequencies (e.g., frequencies that are the human equivalent of marmoset alpha and/or theta frequencies) may be used to evaluate and monitor mental health conditions, efficacy of drug treatment, identification of candidates who may respond to a given anxiolytic drug, indicate when a next dosage may be necessary, etc. For example, alpha and/or theta wave activity may be used to determine that a subject is responsive to an anxiolytic drug, e.g., in response to identifying that alpha and/or theta wave activities increase for a subject within a predetermined amount of time following treatment with a medication being tested.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “control system” on data stored on one or more computer-readable storage devices or received from other sources.

The term “control system” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### CONCLUSION

The various methods and techniques described above provide a number of ways to carry out the invention. Of course, it is to be understood that not necessarily all objectives or advantages described can be achieved in accordance with any particular implementation described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some implementations specifically include one, another, or several features, while others specifically exclude one, another, or several features, while still others mitigate a particular feature by inclusion of one, another, or several advantageous features.

Furthermore, the skilled artisan will recognize the applicability of various features from different implementations. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse implementations.

Although the application has been disclosed in the context of certain implementations, approaches, embodiments, examples, etc., it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

The invention claimed is:

1. A system for evaluating mental health in a subject, the system comprising:
a set of Electroencephalography (EEG) sensors configured to output brain wave data collected from the subject;
a processor; and
logic integrated with and/or executable by the processor, the logic being configured to:
receive, by the processor, brain wave data from the set of EEG sensors, the brain wave data corresponding to a treatment administered to the subject;
evaluate, by the processor, the brain wave data to determine an EEG profile over a period of time, the EEG profile being based on (i) alpha brain wave activity of the subject and/or (ii) theta brain wave activity of the subject, during the period of time;
determine, by the processor, a mental health condition of the subject using the EEG profile, wherein determining the mental health condition of the subject includes:
in response to determining the alpha brain wave activity and/or the theta brain wave activity has decreased below respective threshold activity levels at one or more points over the period of time, identifying the subject as having an anxiety disorder; and
output, by the processor via a user interface, an indication of the mental health condition.

2. The system of claim 1, wherein the treatment is a vasopressin receptor antagonist, wherein determining the mental health condition of the subject includes: determining the subject is a responder to the treatment in response to determining the alpha brain wave activity and/or theta brain wave activity has increased above a threshold activity level after the treatment was administered.

3. (canceled)

4. The system of claim 1, wherein the logic is further configured to: in response to receiving an indication, via a user interface, of the treatment administered to the subject, monitoring subsequent brain wave data received from the EEG sensors; and determining a dosage amount and dosage interval for the treatment based on (i) an amount of increase in alpha brain wave activity and/or theta brain wave activity, and (ii) a point in time that the increase in alpha brain wave activity and/or theta brain wave activity was detected.

5. The system of claim 1, wherein the logic is further configured to: in response to receiving an indication, via a user interface, of the treatment administered to the subject, monitoring subsequent brain wave data received from the EEG sensors; and determining when a next dosage of the treatment should be administered based on decreased alpha wave frequency and/or decreased theta wave frequency following an initial increase in alpha and theta wave frequencies after the treatment was administered t.

6. The system of claim 1, wherein the mental health condition is anxiety and wherein the treatment administered to the subject includes at least one anxiolytic compound.

7. The system of claim 6, wherein the at least one anxiolytic compound is a vasopressin receptor antagonist.

8. The system of claim 1, wherein the set of EEG sensors are disposed in a wearable EEG headset.

9. A method for mental health evaluation of a subject, comprising:
receiving Electroencephalography (EEG) data from a plurality of EEG sensors, the EEG data corresponding to the subject;
processing the EEG data to determine a mental health condition of the subject, the mental health condition indicating the subject has anxiety, wherein the indication of anxiety is based on one or more of the alpha brain wave and the theta brain wave frequencies remaining below a threshold activity level;
wherein the mental health condition of the subject is based on alpha brain wave activity and/or theta brain wave activity.

10. (canceled)

11. The method of claim 9, further comprising: in response to a treatment having a vasopressin receptor antagonist being administered to the subject, determining timing and dosage of the vasopressin receptor antagonist based on a change in one or more of the alpha brain wave activity and the theta brain wave activity.

12-19. (canceled)
